(19) United States
(12) Patent Application Publication
(10) Pub. No.: US 2020/0015722 A1
(43) Pub. Date: Jan. 16, 2020

(54) RESONANCE-BASED PHYSIOLOGICAL MONITORING

(71) Applicant: Massachusetts Institute of Technology, Cambridge, MA (US)

(72) Inventors: Kaustav A. Gopinathan, Brookline, MA (US); Eduardo A. Maury, Miami Beach, FL (US); Dylan V. Neel, Providence, RI (US)

(21) Appl. No.: 16/491,061

(22) PCT Filed: Mar. 6, 2018
(86) PCT No.: PCT/US18/21203

§ 371 Date: Sep. 4, 2019

Related U.S. Application Data
(60) Provisional application No. 62/467,779, filed on Mar. 6, 2017.

(51) Int. Cl. A61B 5/145; A61B 5/00

(52) U.S. Cl. CPC A61B 5/14546; A61B 5/14503; A61B 2562/028; A61B 5/6852; A61B 5/686

(57) ABSTRACT

A passive electronic resonator can be configured with a resonant frequency that changes according to changes in the permittivity of a surrounding environment. A wireless device can then wirelessly measure the formation of a biological deposition such as thrombosis or biofilm on the resonator by wirelessly measuring changes in the resonant frequency reflected in corresponding changes to a driving point impedance for the resonator.

### CROSS-REFERENCE TO RELATED APPLICATIONS

This application is a national stage entry application of International Patent Application No. PCT/US18/21203 filed on Mar. 6, 2018, which claims priority to U.S. Provisional Patent Application No. 62/467,779 filed on Mar. 6, 2017, where the entire contents of each of the foregoing are incorporated herein by reference.

### BACKGROUND

Thrombosis, infection, and the like can compromise the effectiveness of implantable medical devices, leading to decreased effectiveness and worsening patient prognosis. Existing techniques for in vivo measurement of these types of biological depositions typically rely on wired connections, or complex electronic circuitry to power sensors or interpret results. There remains a need for improved techniques to detect the formation of biofilms and the like on implanted medical devices.

### SUMMARY

A passive electronic resonator can be configured with a resonant frequency that changes according to changes in the permittivity of a surrounding environment. A wireless device can then wirelessly measure the formation of a biological deposition such as thrombosis or biofilm on the resonator by wirelessly measuring changes in the resonant frequency reflected in corresponding changes to a driving point impedance for the resonator.

In one aspect, a system disclosed herein includes a medical device and a resonator coupled to the medical device, the resonator including a passive electrical circuit formed of a conductive trace and configured to resonate at a resonant frequency in a radio frequency range, where the resonant frequency varies in response to a change in a permittivity of a material disposed on the resonator in a region adjacent to a portion of the passive electrical circuit.

The medical device may include at least one of a catheter, a prosthetic heart valve, a ventricular assist device, an inferior vena cava filter, and orthopedic prosthetic hardware. The system may further include a biocompatible material disposed about the resonator to separate the conductive trace from a physiological environment. The biocompatible material may include a thin biocompatible coating that does not substantially fill a sensing volume about the resonator. The biocompatible material may have a thickness in a region above the passive electrical circuit less than a second thickness of the conductive trace. The biocompatible material may have a thickness less than about one millimeter. The biocompatible material may form a thin, low-permittivity barrier between the passive electrical circuit and a sensing volume. An exterior surface of the biocompatible material may be formed of a material selected to accumulate a deposition of a predetermined biomaterial at a rate correlated to an accumulation of the predetermined biomaterial on a region of interest on the medical device. The predetermined biomaterial may include at least one of thrombus, pannus, calcification, endothilialization, prosthetic valve endocarditis, a microbial film, and a pathogenic biofilm. The passive electrical circuit may include a planar spiral inductor. The passive electrical circuit may include a two-layer circuit. The passive electrical circuit may include at least one non-planar feature. The conductive trace may be a metal trace formed of at least one of copper, gold, and silver. The conductive trace may be formed of polypyrrole. The system may further include a non-conductive substrate for the conductive trace. The passive electrical circuit may be configured to resonate at two or more different resonant frequencies. The passive electrical circuit may include two or more separate passive electrical circuits, each having a different resonant frequency. The system may further include a reader configured to wirelessly measure one or more resonant frequencies of the resonator. The reader may measure one or more resonant frequencies using at least one of a sinusoidal excitation, a frequency sweep, a broadband excitation, and one or more phase locked loops. The reader may further include a circuit operable to create a time varying electromagnetic field, a resonant frequency detection circuit operable to detect a response of the resonator to the time varying electromagnetic field, including at least one of a phase change and a magnitude change in a driving point impedance for the resonator resulting from a biological deposition on the resonator, and a processor configured to calculate at least one of an extent and a type of the biological deposition based on the response. The reader may be configured to detect two or more resonant frequencies of the resonator and to determine a type of biological material accumulated on the resonator based on changes to one or more resonant frequencies. The reader may be configured to detect two or more resonant frequencies of the resonator and to determine a type of biological material accumulated on the resonator based on changes to one or more of a magnitude and a phase of the driving-point impedance.

In one aspect, a method disclosed herein includes implanting a resonator enclosed in a biocompatible material in a biological medium, the resonator including a passive electrical circuit formed of a conductive trace and configured to resonate at a resonant frequency in a radio frequency range, where the resonant frequency varies in response to a change in a permittivity of a material disposed on the biocompatible material in a region adjacent to a portion of the passive electrical circuit, wirelessly measuring the resonant frequency of the resonator, and detecting a development of a biological deposition on the biocompatible material based on the resonant frequency.

Detecting the development of the biological deposition may include detecting at least one of a thickness and a type of the biological deposition. The method may further include measuring a baseline resonant frequency of the resonator after implanting and before the development of the biological deposition to establish a baseline for detecting the biological deposition. Detecting the development of the biological deposition may include determining an extent of the development based on a change from the baseline resonant frequency to the resonant frequency. The method may further include determining a medical action responsive to the development of the biological deposition. The method may further include creating a model relating changes in the resonant frequency to an extent of the development of the biological deposition. The model may include a patient-specific model. The model may include a model for a particular medical device. The model may include a model for a particular type of material accumulating on the resonator.

In one aspect, a computer program product disclosed herein includes computer executable code embodied in a nontransitory computer readable medium that, when executing on one or more computing devices, performs the steps of creating a model relating changes in a resonant frequency of a resonator to a development of a biological deposition on the resonator, receiving a measurement of the resonant frequency, calculating an extent of the biological deposition on the resonator based on the measurement and the model, and displaying an indication of the extent of the biological deposition. The computer program product may further include code that performs the step of controlling a reader to wirelessly measure the resonant frequency.

### BRIEF DESCRIPTION OF THE FIGURES

FIG. 1A shows a sensor for measuring biological deposition.

FIG. 1B shows an illustrative circuit model of a lumped element resonator used in a sensor.

FIG. 2 shows a sensor for measuring biological deposition.

FIG. 3 depicts a progression of electrical behaviors of a resonator as a biological deposition accumulates in a sensing volume.

FIG. 4 shows a system including a sensor and a reader for measuring biological deposition.

FIG. 5 shows a sensor on a leaflet of a prosthetic heart valve.

FIG. 6 illustrates a clinical management strategy for heart valve thrombosis.

FIG. 7 shows a sensor on a surface of a catheter.

FIG. 8 shows a computer architecture for a biological deposition measurement system.

FIG. 9 shows a method for measuring biological depositions.

FIG. 10 shows a wireless measurement of in vitro clot formation.

### DETAILED DESCRIPTION

The embodiments will now be described more fully hereinafter with reference to the accompanying figures, in which preferred embodiments are shown. The foregoing may, however, be embodied in many different forms and should not be construed as limited to the illustrated embodiments set forth herein.

All documents mentioned herein are hereby incorporated by reference in their entirety. References to items in the singular should be understood to include items in the plural, and vice versa, unless explicitly stated otherwise or clear from the context. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth.

Recitation of ranges of values herein are not intended to be limiting, referring instead individually to any and all values falling within the range, unless otherwise indicated herein, and each separate value within such a range is incorporated into the specification as if it were individually recited herein. The words “about,” “approximately,” “substantially” or the like, when accompanying a numerical value, are to be construed as including any deviation as would be appreciated by one of ordinary skill in the art to operate satisfactorily for an intended purpose. Ranges of values and/or numeric values are provided herein as examples only, and do not constitute a limitation on the scope of the described embodiments. The use of any and all examples or exemplary language (“e.g.,” “such as,” or the like) provided herein, is intended merely to better illuminate the embodiments and does not pose a limitation on the scope of the embodiments or the claims. No language in the specification should be construed as indicating any unclaimed element as essential to the practice of the disclosed embodiments.

In the following description, it is understood that terms such as “first,” “second,” “top,” “bottom,” “above,” “below,” “up,” “down,” and the like, are words of convenience and are not to be construed as limiting terms unless specifically stated.

Unless otherwise explicitly stated or clear from the context, the term “electrically significant” as used herein refers to an electromagnetic wave, field, force, current, or the like that can be detected by the systems described herein, e.g., that may be measured or detected using conventional circuitry and/or any of the circuits or systems described herein. As used herein, a resonator is considered “energized” when an electrically significant current such as a displacement current or a conventional current flows through the conductors or dielectrics of the resonator. As used herein, the term “sensing volume” refers to a volumetric region around the resonator where a change in permittivity of material, such as that which might result from a deposition of a biofilm, can be detected through a change in a resonant frequency of the energized resonator.

In general, the systems and methods described herein are intended to measure an extent of a biological deposition. As used herein, the term “extent” may refer to any one or more properties that might characterize an amount or degree of biological deposition including, without limitation, the mass, volume, thickness, branching geometry, density, complex dielectric constant, and the volume fraction of the cell types that make up the biological deposition, as well as any combination of the foregoing.

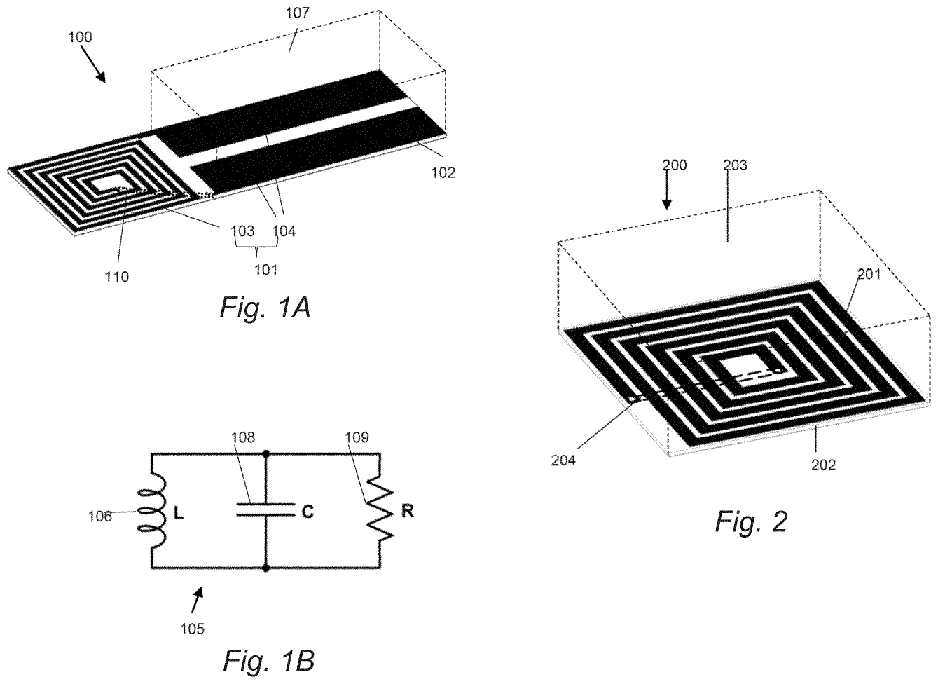

FIG. 1A shows a sensor for measuring a biological deposition. In general, the sensor 100 may be any passive electrical circuit formed, e.g., of a conductive trace of a metal such as copper, gold, silver, or any other metal suitable for use in a resonator as contemplated herein. Non-metallic materials with suitable conductivity may also or instead be used as the conductive trace. For example, conductive plastics such as polypyrrole may be used to form the conductive trace, and may be preferable for certain embodiments such as on the exterior of a catheter. The resonator may be configured to resonate at a resonant frequency in a radio frequency range, with a resonant frequency that varies in response to a change in a permittivity of a material disposed on the resonator in a region adjacent to a portion of the passive electrical circuit, all as more generally described herein. It will be understood that, while radio frequency resonators advantageously present relatively simple architectures and can operate in frequency ranges at which electromagnetic fields can easily penetrate biological tissue (and other materials such as clothing, etc.) present in the context of a medical implant, other resonators may also or instead be employed without departing from the scope of this disclosure, such as waveguides or other microwave resonators or the like.

The illustrative embodiment of the sensor 100 uses a lumped inductor-capacitor resonator geometry. The resonator 101 may rest on a substrate 102, which may include any biocompatible substrate or, where the sensor 100 is otherwise enclosed in a biocompatible housing, jacket, envelope, or the like, or any other substrate suitable for supporting the resonator 101. In general, the substrate 102 may be a non-conductive material, or may be separated from the resonator 101 by a non-conductive material, in order to avoid interfering with an electrical current path (e.g., through a spiral) that encourages resonant behavior of the sensor 100. The resonator 101 may be coated with a thin biocompatible coating such as any of the coatings described herein. The resonator 101 may, for example, be generally divided into two structures illustrated here as a spiral conducting element 103 and a long electrode pair 104. A conductive trace 110 for terminals of the spiral trace may travel on a different circuit layer from the spiral trace, thus forming a two-layer circuit. More generally, the passive electrical circuit of the resonator 101 may include any non-planar feature or combination of features suitable, e.g., for coupling the terminals of a planar spiral inductor or other element of a resonator to facilitate conduction of current therethrough. In another aspect, a waveguide or other high-frequency resonator may have a planar structure containing, e.g., a meander inductor or the like.

FIG. 1B shows an illustrative circuit model of a lumped element resonator. More specifically, FIG. 1B shows a circuit model 105 of the example lumped element resonator 101 of FIG. 1A. A spiral conducting element 103, such as a planar spiral inductor, may be modelled as a lumped inductor 106. When the resonator 101 is energized, a long electrode pair 104 will form an electric field through the dielectric material in the sensing volume 107. A biological material in the sensing volume 107 may have complex dielectric constants with real and imaginary parts. The long electrode pair 104 may therefore be modelled as a lumped capacitor 108 in parallel with a lumped resistor 109. It will be understood, however, that the embodiment shown in FIGS. 1A-1B is for illustrative purposes only, and that alternative physical geometries and lumped-element geometries of the resonator may also or instead be used, and are intended to fall within the scope of this disclosure.

For a lumped inductor-capacitor resonator, the resonant frequency of the circuit is given by:

$${f}_{0} = \frac{1}{\sqrt{LC}} \qquad [ Eq . 1 ]$$

where L is the lumped inductance 106, C is the lumped capacitance 108, and f₀ is the resonant frequency. As biological material is deposited in the sensing volume 107, the fractional compositions and/or permittivities of the materials in the sensing volume 107 are altered, and therefore the value of the lumped capacitance 108 is changed. Thus, the resonant frequency, f₀, of the resonator 101 is also altered. This change in resonant frequency may be measured remotely by a wireless reader such as any of those described herein, and may be used to determine the extent and other properties of the biological deposition.

FIG. 2 shows a sensor for measuring biological deposition. In particular, FIG. 2 shows an alternative illustrative embodiment of a sensor 200 utilizing distributed element resonator geometry. In general, the sensor 200 may include a resonator 201 such as a planar spiral inductor made of a conductive material resting on a substrate 202. A sensing volume 203 above the sensor 200 provides a region in which changes in permittivity can be effectively measured through remote, wireless measurements of driving point impedance as described herein. The sensor 200 may be coated with a thin biocompatible coating. In general, a conductive trace 204 coupling terminals of a spiral trace of the resonator 201 may travel on a different layer from the spiral trace, thus forming a two-layer circuit, or more generally, a circuit having at least one non-planar feature.

In a distributed element geometry (also known as transmission line geometry), the traces of the circuit may be modelled with resistances, capacitances, and inductances that are distributed continuously throughout the circuit. For illustration purposes, the resonator 201 is depicted as a planar spiral, however other geometries may also or instead be employed. In the depicted structure, the traces of the resonator 201 are designed such that they experience a distributed self/mutual inductance along the length of each trace and adjacent traces, and the traces experience a distributed parasitic capacitance between adjacent turns in the spiral, thus providing inductive and capacitive characteristics to form a resonator. It will be appreciated that the design of the resonator 201 in FIG. 2 is provided for illustrative purpose only, and is not intended to limit the scope of this disclosure. Alternative geometries for a distributed element resonator (such as transmission line geometry) are known, and may suitably be adapted for use with the sensors described herein.

In general, distributed element resonators such as the resonator 201 in FIG. 2 may have more than one resonant frequency, and a behavior that is modeled by a system of partial differential equations. As biological deposition accumulates within the sensing volume of such a resonator, one or more of the resonant frequencies of the circuit will be altered in a predictable fashion, which may be determined, e.g., using analytic solutions, computational modelling, experimental results, or combinations of these. The change in one or more of the resonant frequencies may be measured remotely by a wireless reader as described herein, and used to determine the extent and other properties of deposition. While the resonator 201 depicted in FIG. 2 usefully provides multiple resonant frequencies from a single circuit, it will be understood that a passive electrical circuit for a resonator as contemplated herein may also or instead include two or more separate passive electrical circuits, such as circuits positioned to measure biological deposition at different locations or circuits having different resonant frequencies, e.g., e.g., for measuring different types of biological depositions.

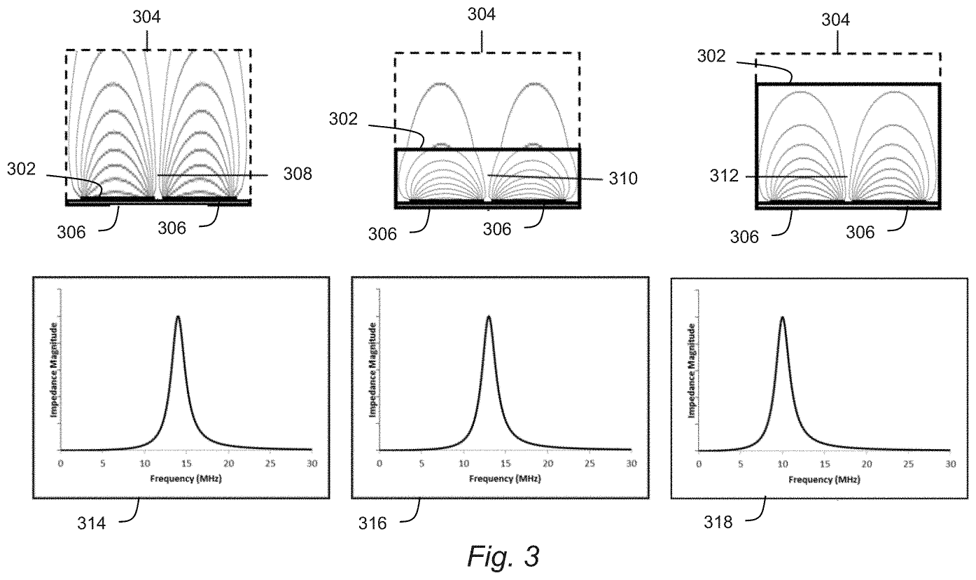

FIG. 3 depicts a progression of electrical behaviors of a resonator as a biological deposition accumulates in a sensing volume. In general, a growing biological deposition 302 may accumulate within a sensing volume 304 over a pair of conductive traces 306. States 308, 310, and 312 depict typical cross sections of the sensing volume before deposition, after moderate deposition, and after high deposition, respectively. The sensing volume 304 may generally be filled with blood or some other normal physiological medium for a medical implant or other device. The biological deposition 302 may include any material that might grow or accumulate on the surface of the sensor within the sensing volume 304. By way of non-limiting examples, the biological deposition 302 may include one or more of thrombus, pannus, calcification, endothilialization, prosthetic valve endocarditis, a microbial film, and a pathogenic biofilm. More generally, any material deposition, growth or other accumulation that changes a permittivity within the sensing volume 304 in a manner having an electrically significant effect on a resonant response of the resonator may be measured using the devices, systems, and methods described herein.

In the example of FIG. 3, the biological deposition 302 accumulates over a pair of lumped capacitor traces in a lumped inductor-capacitor resonator configuration, however it will be appreciated that a similar phenomenon will take place across traces in distributed element resonators as well. Since a normal medium in the sensing volume 304 has a different permittivity from the biological deposition 302, as material is deposited in the sensing volume 304 over the traces 306, the equipotential lines of a field within the sensing volume 304 are altered over the course of deposition, and more particularly, altered in a manner that facilitates remote sensing of corresponding changes in a driving point impedance as contemplated herein. This phenomenon is also illustrated by a sequence of illustrative frequency response graphs 314, 316, and 318, each illustrating a different peak for the driving point impedance of the sensor at a corresponding accumulation of the biological deposition 302. Further, it will be understood that a change in permittivity, as measured by the sensors described herein, may result from changes within the sensing volume 304 as one material displaces another. That is, without any change in permittivity of two different materials, the permittivity of a region around a sensor may change as one material, e.g., the biological deposition 302, displaces another, e.g., normal tissue or bodily fluid.

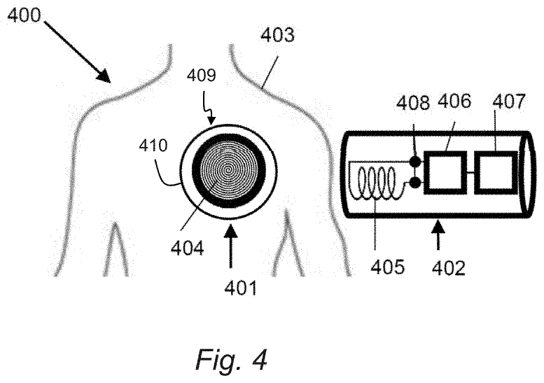

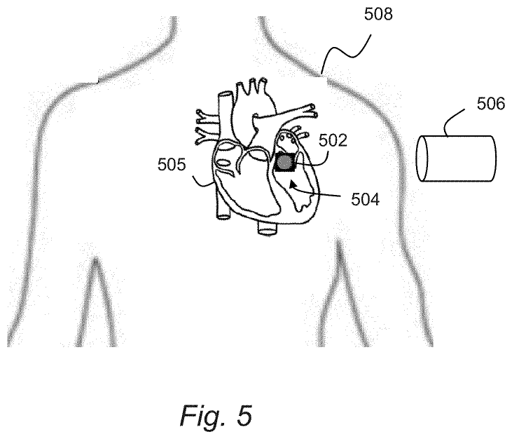

FIG. 4 shows a system including a sensor and a reader for measuring biological deposition. In the system 400, a sensor 401 may be implanted into a human subject 403, either alone or in combination with another medical device or implant to be monitored with the sensor 401. The sensor 401 may generally contain a resonator 404, such as any of the resonators described herein. As biological deposition accumulates within a sensing volume of the sensor 401, one or more of the resonant frequencies of the resonator 404 may be altered. The reader 402 may be positioned over the sensor 401 in any location and orientation suitable for detecting changes in the resonant frequency using techniques contemplated herein.

Although the reader 402 is not in electrical contact with the resonator 404, electrical energy may be wirelessly transmitted between the reader 402 and the resonator 404, thereby allowing the resonator 404 to be energized by the reader 402 and the reader 402 to measure one or more resonant frequencies of the resonator 404. This transfer of electrical energy may be achieved through electrical coupling (generally where energy is transmitted through changes in the electric field), magnetic coupling (generally where energy is transmitted through changes in the magnetic field), electromagnetic coupling (generally where energy is transmitted through both electric and magnetic fields), or any combination of these. Through the coupling phenomenon between the reader 402 and the resonator 404, the reader 402 may remotely determine resonant properties of the sensor 401 without electrical contact.

The reader 402 may, for example, be magnetically coupled to the resonator 404. In this example, the reader 402 may include a circuit 405 such as an inductive coupling coil, resonant frequency detecting circuitry 406, and a processor 407. The processor 407 may be configured to drive a time varying current through the circuit 405 to generate a time varying magnetic field that produces an electromotive force in the resonator 404. Similarly, current in the resonator 404 may generate an electromotive force across the terminals 408 of the circuit 405, or across a separate inductive coupling coil or other circuit wirelessly coupled to the resonator 404 through an electromagnetic field that permits detection of a resonant frequency of the resonator 404. Due to this magnetic coupling phenomenon (or other electromagnetic field coupling, where appropriate), the current-voltage relationship across the circuit 405 is influenced by the impedance of the resonator. The circuit 405 and the resonator 404 may therefore be lumped into a single port equivalent and represented by a frequency-dependent driving-point impedance (referred to herein as the “driving-point impedance”) across the terminals 408. The driving-point impedance across the terminals 408 may have local extrema in magnitude and/or unique values of phase at the resonant frequencies of the resonator 404, despite the lack of physical contact between the resonator 404 and the reader 402.

These properties may be exploited by the resonant frequency detecting circuitry 406 to measure one or more resonant frequencies of the sensor 401 and determine corresponding biological depositions. This may include changes in the phase and/or magnitude of the driving point impedance in response to a biological deposition on the sensor 401. These changes may be measured using a number of different electrical techniques including, but not limited to, frequency sweeping, response to broadband excitation, and phase-locked loops.

In a frequency sweeping technique, an oscillator is swept over a range of frequencies and is used to excite the reader circuitry. At each frequency the impedance of the driving-point impedance is measured. The processor 408 may then evaluate one or more resonant frequencies based on extrema of the impedance magnitude or a specific impedance phase. This sweeping process may additionally be repeated over successively smaller bandwidths to increase frequency resolution and signal-to-noise ratio to the desired level of accuracy.

In a high bandwidth signal response technique, a high bandwidth signal containing a range of discrete or broadband frequencies may be directed toward the sensor 401, and driving-point impedance across a corresponding frequency range may be measured. The processor 407 may then evaluate the resonant frequency or frequencies.

In the phase-locked loop technique, a negative feedback loop may be used to tune the resonant frequency of an oscillator driving the driving-point impedance. The feedback loop may generally tune the oscillator to a frequency where a certain condition of the driving-point impedance is met, such as reaching a specific impedance phase value or maximization/minimization of the impedance magnitude. The processor 407 may then determine a resonant frequency from the tuning signal controlling the oscillator.

In one aspect, the reader 402 may include a circuit (such as an inductive coupling circuit) operable to create a time varying electromagnetic field, a resonant frequency detection circuit operable to detect a response of the resonator to the time varying electromagnetic field, including at least one of a phase change and a magnitude change in a driving point impedance for the resonator resulting from a biological deposition on the resonator, and a processor configured to calculate at least one of an extent and a type of the biological deposition based on the response.

While a sensor with a single resonant frequency may be useful in many applications, multiple frequencies may also or instead be employed, e.g., to detect the presence of different types of materials, or to distinguish between multiple materials that might be present, either alone or in combination, as a biological deposition on the sensor. Thus, the reader 402 may be configured to detect two or more resonant frequencies of the resonator 404 and to determine a type of biological material accumulated on the resonator 404 based on a magnitude and a phase of each of the two or more resonant frequencies.

In another aspect, a single sinusoidal excitation or the like may be used to measure a response of the resonator 404 to a biological deposition. For example, a phase of the driving point impedance of the resonator 404 may change in addition to or instead of the magnitude, thus permitting detection of some changes based on the phase shift in a single sinusoidal excitation.

In general, the driving point impedance may be a mathematical representation of an input impedance for the resonator circuit in the frequency domain, and may be evaluated using, e.g., a complex ratio of an applied voltage to a resulting current or vice versa. While this provides a useful analytical framework for measuring changes in resonator properties resulting from biological depositions, it will be understood that other similar and equivalent measurements may also or instead be used. All such equivalent techniques for wirelessly sensing changes in behavior of the resonator 404 should be understood as measuring a driving point impedance as contemplated herein, and the reader 402 may usefully employ any such techniques without departing from the scope of this disclosure.

The construction and operation of circuits to measure driving point impedance using the foregoing techniques is well understood in the art, and further details are omitted here. Furthermore, different materials will have different effects on resonators at different frequencies. As such, a detailed elaboration of all such combinations and uses of multi-frequency measurements is omitted here, except to note that one of ordinary skill in the art may readily construct and calibrate a range of multi-frequency resonator structures to determine extents and types of biological depositions as contemplated herein. In a general embodiment, the processor 407 may use one or more measured resonant frequencies along with previous measurements, calibration measurements, computational models, empirical models, and/or analytic models to determine and track the extent and other properties of biological deposition(s) over time.

In general, the sensor 401 may include a shell 410 such as a sleeve, enclosure, film, or the like, of a biocompatible material disposed about the resonator 404 to separate the conductive trace and/or other components of the resonator 404 from a physiological environment within the subject 403. For a standalone sensor 401, this may include a complete volumetric enclosure. When the sensor 401 is coupled to or otherwise associated with another medical device, the shell 410 may secure the resonator 404 to the medical device, e.g., in a manner that encloses the resonator 404 between the medical device and the shell 410. Thus, the shell 410 may generally be disposed about some or all of the resonator 404, in any manner suitable for separating the resonator 404 from a physiological environment in which the sensor 401 is deployed.

The shell 410 may have any construction, and be formed of any biocompatible material consistent with the use of the sensor 401 to measure biological deposition as contemplated herein. As used herein, the term “biocompatible” means generally not harmful to living tissue or other structures within a subject such as a human patient, unless a different meaning is explicitly provided or otherwise clear from the context.

In order to facilitate operation of the sensor 401, the shell 410 may include a biocompatible material forming a thin, low-permittivity barrier between the passive electrical circuit and a sensing volume. For example, the shell 410 may usefully be formed of a biocompatible coating that does not substantially fill a sensing volume about the resonator 404. In general, the thickness of the shell 410 may encroach upon the sensing volume in regions adjacent to the resonator 404—regions with the greatest sensitivity to biological depositions. Thus, the shell 410 may advantageously be made as thin as possible, consistent with maintaining a separation between the resonator 404 and the physiological environment, in order to preserve sensitivity of the sensor 401 to conditions such as accumulations of biological depositions. In another aspect, the biocompatible material of the shell 410 may have a thickness in a region above the passive electrical circuit less than a second thickness of the conductive trace, or a thickness of about one millimeter or less.

In many deployments, the purpose of the sensor 401 is to detect a potentially harmful accumulation of a biological deposition on a medical device or implant. Thus, the sensor 401 will preferably accumulate a biological deposition (or a proxy for the biological deposition) in the sensing volume at a rate equal to or correlated to a rate at which the same biological deposition accumulates on the medical device or a portion thereof. To this end, an exterior surface 409 of the biocompatible material of the shell 410 may be formed of a material selected to accumulate a deposition of a predetermined biomaterial at a rate correlated to an accumulation of the predetermined biomaterial on a region of interest on the medical device. This may be the same material as the exterior of the medical device, or a material with similar interactions with the predetermined biomaterial(s). By way of non-limiting examples, the predetermined biomaterial may include one or more of thrombus, pannus, calcification, endothilialization, prosthetic valve endocarditis, a microbial film, and a pathogenic biofilm.

It will also be appreciated that some biological depositions may be beneficial, such as a less thrombogenic formation of pannus or the like on certain devices, and the sensor 401 described herein may also or instead be used to measure such beneficial accumulations of biological depositions without departing from the scope of this disclosure.

FIG. 5 shows a sensor on a leaflet of a prosthetic heart valve. In this embodiment, a sensor 502 is placed on the surface of a mechanical heart valve 504 for a heart 505 and a handheld reader 506 may be positioned over the chest of a human subject 508 in order to sense biological deposition on the sensor 502. Because thrombosis is a frequent complication of mechanical heart valves and may lead to adverse outcomes after placement of a mechanical heart valve 504 in a patient, it may be advantageous to track thrombus formation on the valve 504. In this example, the normal medium is blood, and the biological deposition includes thrombus. At any suitable intervals, such as fixed times each day (e.g. once per day), week, or the like, the subject 508, or an assistant, technician, or other medical professional or caregiver, may position the reader 506 over the subject 508 and measure thrombus formation via changes to the resonant frequency of the sensor 502. More specifically, using previous measurements, calibration measurements, theoretical models, empirical models, and so forth, the reader 506 may process measurements of driving point impedance and use this data to track biological depositions such as thrombus formation over time, thereby providing information to guide treatment. Although this example uses blood as the normal medium and thrombus as the deposited biological material, will be understood that similar techniques may be employed to other combinations of normal media and deposited biological material over a heart valve, e.g., for endocarditis and so forth.

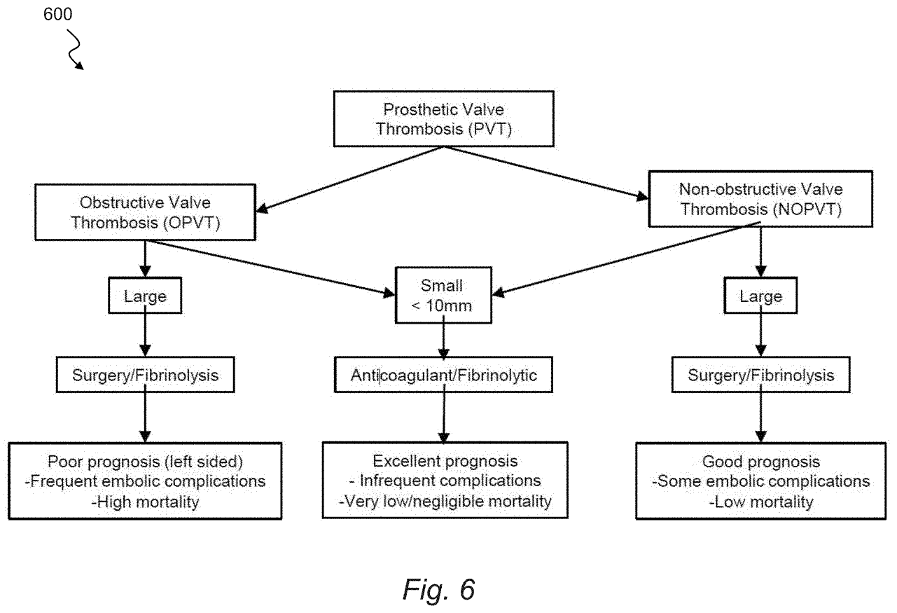

FIG. 6 illustrates a clinical management strategy for heart valve thrombosis. In this overall strategy 600, detection and quantification of thrombosis using the techniques described herein may, for example, be used to direct more patients into the small NOPVT/OPVT (non-obstructive/obstructive prosthetic heart valve thrombosis) treatment regime based on actual measurements of thrombus formation, which may advantageously reduce the number of costly and risky heart valve replacement surgeries, lower the rate of embolic complications associated with fibrinolysis of large thrombi, and improve patient prognosis.

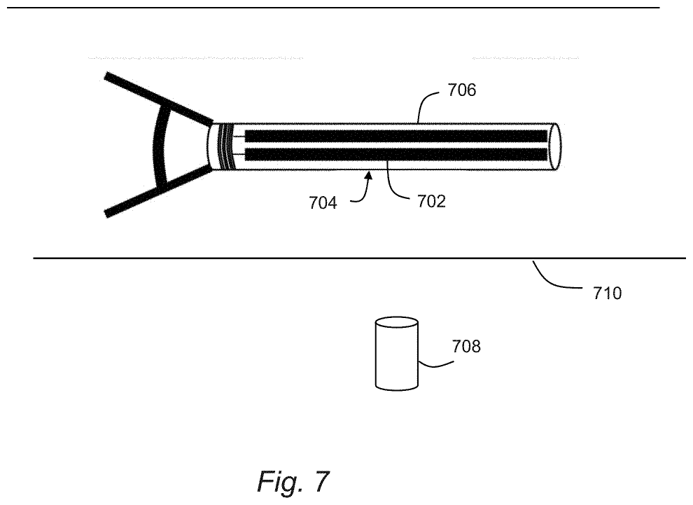

FIG. 7 shows a sensor on a surface of a catheter. In general, a sensor 702 such as any of the sensors described herein may be disposed on a surface 704 of a medical device 706 such as on a luminal or outer surface of an intravenous medical catheter. A reader 708, such as any of the readers described herein, may be positioned in a suitable location to measure resonance of the sensor 702 and used to measure a biological deposition on the sensor 702 through tissue 710 of a subject as generally described herein. Venous, arterial, and urinary catheters provide routes for infective agents to enter the body and therefore may increase the risk of infections. Since many infections from a catheter can also form biofilms over a surface of the catheter, it may be advantageous to track the formation of biofilms on the surface of a catheter as a proxy for the presence of infection. In this example, the normal medium is blood, and the deposited biological material is bacterial/fungal biofilm. At fixed times each day (e.g. twice per day) while the catheter is inserted into the tissue 710 of a subject, the reader 708 may be positioned over the sensor 702 and used to detect one or more resonant frequencies of the sensor 702. Using previous measurements, calibration measurements, and theoretical or empirical models, the reader 708 may then process these measurements to track the formation of biofilm over time, thereby providing information to a physician to guide treatment. Although this example uses blood as the normal medium and pathological biofilm as the deposited biological material, it is understood that a similar description applies to other combinations of normal media and deposited biological material over a catheter.

Using a sensor 702 on a medical device 706 such as a catheter may permit measurement of biofilm formation on catheters at risk for infection, such as in patients with long-term central venous catheters that are at risk. This may include, for example, cancer patients, transplant patients, and/or long-term hemodialysis patients, among others. This information may be used, for example, to facilitate monitoring of microbial deposition, thus providing information for healthcare providers to adjust antimicrobial therapies such as antibiotics to improve patient outcomes.

While the foregoing description identifies mechanical heart valves and catheters, it will be understood that any implantable medical device, surgical tool, or the like may usefully be instrumented to detect biological depositions as described herein. Thus, for example, the medical device may include a prosthetic heart valve, a catheter, a ventricular assist device, an inferior vena cava filter, a stent, a pacemaker, an implanted nerve stimulation device, an ostomy bag, a peripheral vascular device, orthopedic prosthetic hardware, or any other prosthetic implant, surgical tool, or other medical device or the like that might accumulate a biological deposition while implanted in a patient.

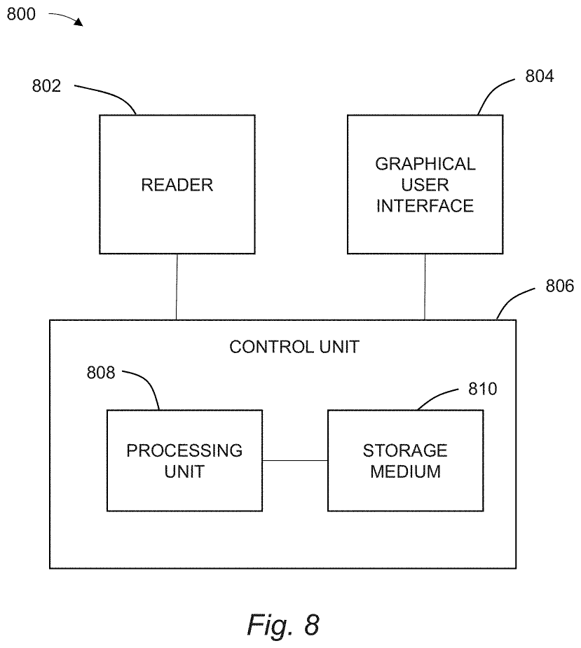

FIG. 8 shows a computer architecture for a biological deposition measurement system. In general, the system 800 may include a reader 802 such as any of the wireless readers described herein, a graphical user interface 804, and a control unit 806 in communication with the reader 802 and the graphical user interface 804.

The control unit 806 may include a processing unit 808 such as any processor, combination of processors or other processing circuitry, and the like. The control unit 806 may also include a non-transitory, computer-readable storage medium 810 for storing, inter alfa, instructions executed by one or more processors of the processing unit 808. In use, the reader 802 can be positioned to capture a measurement from a sensor such as any of the sensors described herein, and the control unit 806 can generate control instructions for operating the reader 802, capturing feedback from the sensor, and processing results to determine changes in biological deposition on the sensor. The control unit 806 may, for example, include a desktop, a laptop, a tablet, a mobile phone, or any other computing device.

The storage medium 810 may store data captured from the sensor through the reader 802, as well as any models, calibration data, or the like useful for interpreting the sensed data and determining the extent and/or type of biological deposition(s) on a sensor. The storage medium 810 may be integrally built into the reader 802, e.g., so that the reader 802 can operate as a standalone device. Additionally, or alternatively, the storage medium 810 may include external storage, such as in a desktop computer, network-attached storage, or other device, e.g., to log raw sensor/reader data, processed results, and so forth. In one aspect, data may be wirelessly transmitted from the reader 802 to the storage medium 810 to facilitate wireless operation of the reader 802. Wired communications may also or instead be used to transmit data from the reader 802 to the storage medium 810.

The graphical user interface 804 can be a graphical display of any known type or construction (e.g., a computer monitor associated with a desktop computer and/or a laptop computer) and can be in wired or wireless communication with the control unit 806 and/or the reader 802. Raw data, results of calculations, and so forth may be displayed on the graphical user interface 804 in any suitable format, along with menus, controls, and so forth for controlling operation of the system 800. In one aspect, the graphical user interface 804 can be integrated into the reader 802 to provide an integrated, hand-held, and/or portable device.

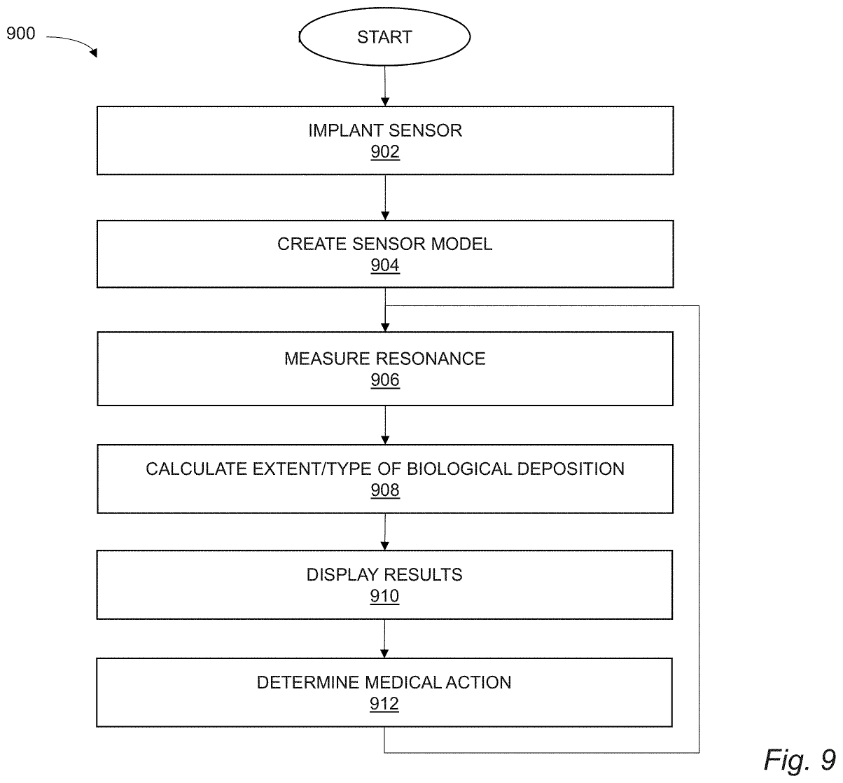

FIG. 9 shows a method for measuring biological depositions using the systems described herein.

As shown in step 902, the method 900 may begin with implanting a sensor, such as any of the resonators described herein, in a biological medium such as a patient. This may, for example, include implanting a sensor having a resonator enclosed in a biocompatible material, where the resonator includes, e.g., a passive electrical circuit formed of a conductive trace and configured to resonate at a resonant frequency in a radio frequency range, and where the resonant frequency varies in response to a change in a permittivity or composition of a material disposed on the biocompatible material in a region adjacent to a portion of the passive electrical circuit. More generally, this may include implanting one or more of any of the sensors and resonators described herein, as well as implanting, e.g., an associated medical device that might accumulate a biological deposition while implanted.

As shown in step 904, the method 900 may include creating a sensor model 904, such as by creating a model relating changes in a resonant frequency of a resonator to a development of a biological deposition (or an extent of the development of the biological deposition) on the resonator as generally described herein. The model may be any analytical model, empirical model, calibration model, or the like. The model may, for example, be based on the geometry of a sensor/resonator, the type of biological deposition to be measured, and so forth. This may also or instead include specific calibration data or other modelling or measurements acquired for a specific patient, e.g., after the sensor has been implanted. Thus, in one aspect, the model may include a patient-specific model based on measurements taken after the sensor is implanted in the patient.

In another aspect, the model may be a model for a particular medical device. This may, for example, include capturing calibration data for a particular medical device after implant. This may also or instead include creating a theoretical or analytical model to detect a particular type of biological deposition related to the particular medical device. For example, as described above a mechanical heart valve may usefully be instrumented to detect a blood clot or thrombus forming on the surface, and a detection model may be created for the sensor to specifically detect this type of biological deposition on a sensor and/or an associated medical device. The model may also or instead include a model for a particular type of material accumulating on a resonator of a sensor. As noted above, different biological depositions may have different properties, e.g., different permittivities, and a sensor such as a multi-frequency sensor may usefully be adapted with a suitable model to discriminate among different types of biological depositions based on these properties instead of, or in addition to, detecting an extent of the biological depositions.

In another aspect, creating the sensor model may include measuring a baseline resonant frequency of the resonator after implanting and before the development of the biological deposition in order to establish a baseline for detecting the biological deposition. For such a model, subsequently detecting the development of the biological deposition may include determining an extent of the development based on a change from the baseline resonant frequency to a current measurement of the resonant frequency.

As shown in step 906, the method 900 may include wirelessly measuring the resonant frequency of the resonator, such as by controlling a reader to wirelessly measure the resonant frequency by measure the driving point impedance of a sensor, and/or by receiving a measurement of the resonant frequency from the sensor/reader based on the driving point impedance.

As shown in step 908, the method 900 may include calculating an extent or type of biological deposition. For example, this may include detecting a development of a biological deposition on the biocompatible material based on the phase and/or magnitude of the driving point impedance for the resonant frequency, or more specifically detecting at least one of a thickness and a type of the biological deposition. This may include applying the model developed in step 904. For example, this may include calculating an extent of the biological deposition on the resonator based on the measurement(s) from the reader/sensor and based upon the model.

As shown in step 910, the method 900 may optionally include displaying results, such as an extent (e.g., thickness, volume, etc.) of biological deposition, a type of biological deposition, and so forth. Where results are uncertain or ambiguous, this may also or instead include displaying a confidence level or other statistical (or other measure) of expected accuracy or the like. Thus, for example, this may include displaying a percentage confidence that a type of biological deposition has been correctly identified based on a multi-frequency measurement obtained from one or more sensors.

As shown in step 912, the method 900 may optionally include determining a medical action responsive to the development of the biological deposition. Specific strategies for addressing thrombosis on prosthetic heart valves are described above with reference to FIG. 6, for example. It will be readily appreciated that a particular course of action may depend on the particular implant, sensor, patient, and/or patient condition in question, as well as the extent and type of biological deposition detected by a sensor. In general, however, the systems and methods described herein can advantageously facilitate clinical decision-making by providing actual measurements of biological depositions on implanted devices.

After a measurement has been taken, and where appropriate, after any post-measurement steps have been completed, the method may return to step 906 where an additional resonance measurement may take place. This may be repeated any number of times on any suitable schedule as necessary or helpful for measuring and acting on accumulations of biological depositions as contemplated herein. In one aspect, this may include periodic measurements associated with a course of treatment, such as measurements once or twice per day. In another aspect, this may include multiple concurrent measurements in order to verify a current reading.

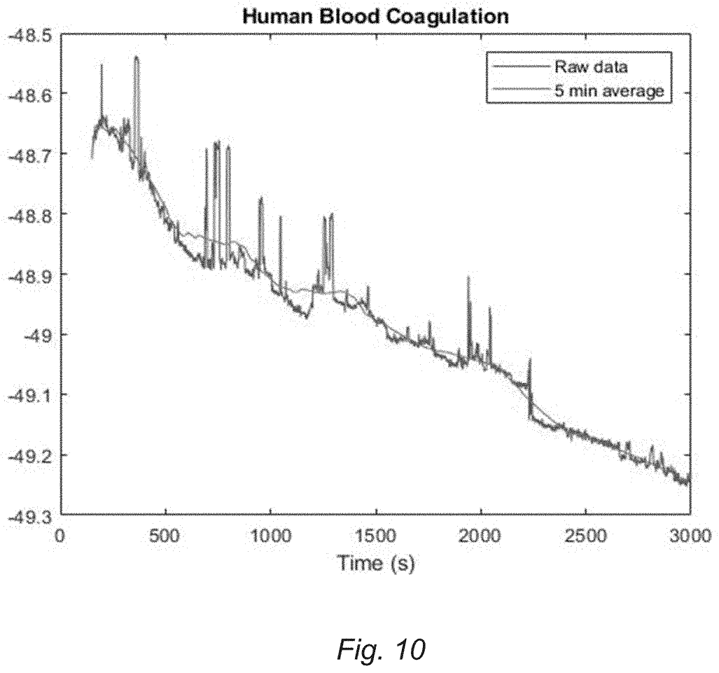

FIG. 10 shows a wireless measurement of in vitro clot formation. In order to demonstrate the efficacy of the disclosed techniques, a planar spiral sensor was placed in a petri dish of unclotted blood at room temperature. As depicted, in less than ten minutes (or, less than five hundred seconds on the x-axis), a measurable change in the phase of the driving point impedance (on the y-axis) occurred for the sensor in response to a single sinusoidal excitation. This change continued to progress generally linearly as a clot formed on the sensor over time within the petri dish.

The above systems, devices, methods, processes, and the like may be realized in hardware, software, or any combination of these suitable for a particular application. The hardware may include a general-purpose computer and/or dedicated computing device. This includes realization in one or more microprocessors, microcontrollers, embedded microcontrollers, programmable digital signal processors or other programmable devices or processing circuitry, along with internal and/or external memory. This may also, or instead, include one or more application specific integrated circuits, programmable gate arrays, programmable array logic components, or any other device or devices that may be configured to process electronic signals. It will further be appreciated that a realization of the processes or devices described above may include computer-executable code created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software. In another aspect, the methods may be embodied in systems that perform the steps thereof, and may be distributed across devices in a number of ways. At the same time, processing may be distributed across devices such as the various systems described above, or all of the functionality may be integrated into a dedicated, standalone device or other hardware. In another aspect, means for performing the steps associated with the processes described above may include any of the hardware and/or software described above. All such permutations and combinations are intended to fall within the scope of the present disclosure.

Embodiments disclosed herein may include computer program products comprising computer-executable code or computer-usable code that, when executing on one or more computing devices, performs any and/or all of the steps thereof. The code may be stored in a non-transitory fashion in a computer memory, which may be a memory from which the program executes (such as random-access memory associated with a processor), or a storage device such as a disk drive, flash memory or any other optical, electromagnetic, magnetic, infrared or other device or combination of devices. In another aspect, any of the systems and methods described above may be embodied in any suitable transmission or propagation medium carrying computer-executable code and/or any inputs or outputs from same.

The method steps of the implementations described herein are intended to include any suitable method of causing such method steps to be performed, consistent with the patentability of the following claims, unless a different meaning is expressly provided or otherwise clear from the context. So, for example performing the step of X includes any suitable method for causing another party such as a remote user, a remote processing resource (e.g., a server or cloud computer) or a machine to perform the step of X. Similarly, performing steps X, Y and Z may include any method of directing or controlling any combination of such other individuals or resources to perform steps X, Y and Z to obtain the benefit of such steps. Thus, method steps of the implementations described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity, and need not be located within a particular jurisdiction.

It should further be appreciated that the methods above are provided by way of example. Absent an explicit indication to the contrary, the disclosed steps may be modified, supplemented, omitted, and/or re-ordered without departing from the scope of this disclosure.

It will be appreciated that the devices, methods and systems described above are set forth by way of example and not of limitation. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context. Thus, while particular embodiments have been shown and described, it will be apparent to those skilled in the art that various changes and modifications in form and details may be made therein without departing from the spirit and scope of this disclosure and are intended to form a part of the invention as defined by the following claims.

1. A system comprising:
a medical device; and
a resonator coupled to the medical device, the resonator including a passive electrical circuit formed of a conductive trace and configured to resonate at a resonant frequency in a radio frequency range, wherein the resonant frequency varies in response to a change in a permittivity of a material disposed on the resonator in a region adjacent to a portion of the passive electrical circuit.

2. The system of claim 1 wherein the medical device includes at least one of a catheter, a prosthetic heart valve, a ventricular assist device, an inferior vena cava filter, and orthopedic prosthetic hardware.

3. The system of claim 1 further comprising a biocompatible material disposed about the resonator to separate the conductive trace from a physiological environment.

4. The system of claim 3 wherein the biocompatible material includes a thin biocompatible coating that does not substantially fill a sensing volume about the resonator.

5. The system of claim 3 wherein the biocompatible material has a thickness in a region above the passive electrical circuit less than a second thickness of the conductive trace.

6. The system of claim 3 wherein the biocompatible material has a thickness less than about one millimeter.

7. The system of claim 3 wherein the biocompatible material forms a thin, low-permittivity barrier between the passive electrical circuit and a sensing volume.

8. The system of claim 3 wherein an exterior surface of the biocompatible material is formed of a material selected to accumulate a deposition of a predetermined biomaterial at a rate correlated to an accumulation of the predetermined biomaterial on a region of interest on the medical device.

9. The system of claim 8 wherein the predetermined biomaterial includes at least one of thrombus, pannus, calcification, endothilialization, prosthetic valve endocarditis, a microbial film, and a pathogenic biofilm.

10. The system of claim 1 wherein the passive electrical circuit includes a planar spiral inductor.

11. The system of claim 1 wherein the passive electrical circuit includes a two-layer circuit.

12. The system of claim 1 wherein the passive electrical circuit includes at least one non-planar feature.

13. The system of claim 1 wherein the conductive trace is a metal trace formed of at least one of copper, gold, and silver.

14. The system of claim 1 wherein the conductive trace is formed of polypyrrole.

15. The system of claim 1 further comprising a non-conductive substrate for the conductive trace.

16. The system of claim 1 wherein the passive electrical circuit is configured to resonate at two or more different resonant frequencies.

17. The system of claim 1 wherein the passive electrical circuit includes two or more separate passive electrical circuits, each having a different resonant frequency.

18. The system of claim 1 further comprising a reader configured to wirelessly measure one or more resonant frequencies of the resonator.

19. The system of claim 18 wherein the reader measures one or more resonant frequencies using at least one of a sinusoidal excitation, a frequency sweep, a broadband excitation, and one or more phase locked loops.

20. The system of claim 18, the reader further comprising:
a circuit operable to create a time varying electromagnetic field;
a resonant frequency detection circuit operable to detect a response of the resonator to the time varying electromagnetic field, including at least one of a phase change and a magnitude change in a driving point impedance for the resonator resulting from a biological deposition on the resonator; and
a processor configured to calculate at least one of an extent and a type of the biological deposition based on the response.

21. The system of claim 18 wherein the reader is configured to detect two or more resonant frequencies of the resonator and to determine a type of biological material accumulated on the resonator based on changes to one or more resonant frequencies.

22. The system of claim 18 wherein the reader is configured to detect two or more resonant frequencies of the resonator and to determine a type of biological material accumulated on the resonator based on changes to one or more of a magnitude and a phase of a driving-point impedance.

23-33. (canceled)
